(19) United States
(12) Patent Application Publication
(10) Pub. No.: US 2024/0091190 A1
(43) Pub. Date: Mar. 21, 2024

(54) SKIN CREAM FOR REDUCING BED SORES

(71) Applicant: Elsia Kelly, Jamaica, NY (US)

(72) Inventor: Elsia Kelly, Jamaica, NY (US)

(21) Appl. No.: 17/946,067

(22) Filed: Sep. 16, 2022

(51) Int. Cl. A61K 31/235; A61K 9/00; A61K 9/107; A61K 31/355; A61K 33/30; A61K 47/06; A61K 47/10; A61K 47/26; A61K 47/32; A61K 47/44

(52) U.S. Cl. CPC A61K 31/235; A61K 9/0014; A61K 9/107; A61K 31/355; A61K 33/30; A61K 47/06; A61K 47/10; A61K 47/26; A61K 47/32; A61K 47/44

(57) ABSTRACT

A composition for external use that is used to heal and prevent wounds is described. The composition is used to prevent bedsores. The composition includes a thickening agent, an anti-fungal agent, an emulsifier, a glycerol, a skin protectant, a petroleum jelly, an oil, and at least one vitamin.

### FIELD OF THE EMBODIMENTS

The field of the invention and its embodiments relate to a composition to prevent bed sores and a method to generate the composition.

### BACKGROUND OF THE EMBODIMENTS

Bedsores, or pressure ulcers and decubitus ulcers, are injuries to skin and underlying tissue resulting from prolonged obstruction of the peripheral blood vessels of the tissue by compression from the contact surface of the body, resulting in necrosis of the tissue. Bedsores most often develop on skin that covers bony areas of the body, such as the heels, ankles, hips and tailbone. Symptoms of bedsores include: unusual changes in skin color or texture, swelling, pus-like draining, and tenderness, etc.

The three primary contributing factors for bedsores are: constant pressure on any part of one's body that lessens the blood flow to tissues, friction that occurs when the skin rubs against clothing or bedding, and shear that occurs when two surfaces move in the opposite direction. People most at risk of bedsores are those with a medical condition that limits their ability to change positions or those who spend most of their time in a bed or chair. Therefore, elderly persons or long-term care patients who are lying on bedding for a long period of time tend to have bedsores, which is a problem in nursing homes.

Numerous categories of bedsores exist, based on their depth, severity and other characteristics. The degree of skin and tissue damage ranges from red, unbroken skin to a deep injury involving muscle and bone. Specifically, the stages of bedsores are as follows: Stage I, Stage II. Stage III, and Stage IV. In Stage I, the bedsore is superficial, accompanied by redness that does not subside after pressure is relieved. This stage is visually similar to reactive hyperemia (i.e., excessive redness) in skin after prolonged application of pressure. In Stage II, the bedsore includes damage to the epidermis extending into, but no deeper than the dermis. A Stage II bedsore may be termed a blister or abrasion. In Stage III, the bedsore may extend into subcutaneous tissue. In this stage, the blood supply is relatively poor and can be difficult to heal. There may also be subsurface damage that is more extensive than appears on the surface. In Stage IV, the bedsore extends into the muscle, tendon or even bone. Complications of bedsores include: cellulitis, bone and joint infections, cancer, and sepsis.

Strategies to reduce the possibility of developing bedsores includes: taking good care of ones skin, maintaining good nutrition and fluid intake, quitting smoking, managing stress, and exercising daily. Regarding skincare, the skin must be kept clean and dry and must be protected, at friction points, by use of talcum powder. Other suggestions regarding skin care include the use of therapeutic agents or lotions, such as ointments containing bromelain and bucladesine Na. However, all of the aforementioned therapeutic agents are known to have side effects, such as bleeding, and may be painful for the patient to apply. Also, bedsore spray including benzoin may be used on the bedsore, yet may result in irritation, burning, or redness of the skin. In other instances, severe allergic reactions may occur with the user of bedsore sprays, with symptoms including a rash, itching and/or swelling of the face, tongue and/or throat, severe dizziness, and trouble breathing.

Therefore, the current treatment of bedsores depends on the natural healing power of the patient. Thus, a need exists for an improved composition to prevent bedsores that has reduced negative side effects.

Review of related technology:

WO 2019/009628 A9 and KR 1020190005764 A relate to a composition for relieving and treating burns and bedsores. The composition includes an animal lung tissue extract as an active ingredient. Since the composition exhibits effectiveness as a material related to skin wound healing and exhibits an industrially applicable effect associated with the treatment of burns and bedsores, the composition can be used as a therapeutic agent for said diseases.

CN 107550962 A relates to a cream composition for sores.

CN 103830303 B relates to compositions of pre-counteracting bedsores. The composition contains chamomile extract, zinc pyrrolidone carboxylate, and bisabolol. In examples, the composition can also contain vitamin C, D-panthenol, and Herba Cymbopogonis Citrari derivant, etc.

CN 105381366 A relates to a traditional Chinese medicine composition for treating bedsores and a method for preparing the composition. The composition can: promote blood circulation, promote tissue regeneration, improve microcirculation of the wound surface, accelerate lesion tissue repair, and enhance the organism immune function, etc. The ointment is prepared from radix astragali, radix angelica sinensis, rheum officinale, wild chrysanthemums, Sophora flavescens, thunberg fritillary bulb and the like, aerosol is prepared from ajuga pantantha, galangal, geranium wilfordii and the like.

CN 105055607 A relates to traditional Chinese medicine composition for treating qi-blood deficiency type bedsores during clinical care. The traditional Chinese medicine composition is mainly prepared from nine raw medicinal materials, namely, evodia leaves, ephedra herbs, herbs of erectspine sanicle, garden burnet roots, atrina glass, liquorice roots, milkvetch roots, Chinese angelica and slender dutchmanspipe roots, in certain parts by weight. The traditional Chinese medicine composition has effects of tonifying qi-blood, clearing heat, removing toxicity, promoting detoxication and promoting tissue regeneration, can support healthy qi and realizes vigorous qi-blood and internal nourishing of internal organs; meanwhile, skin and hair are nourished externally, microcirculation around wound surfaces is improved, regional blood flow is increased, inflammatory edema as well as tissue ischaemia and hypoxia are alleviated, inflammatory exudation is reduced, absorption and dissipation of inflammations are promoted, wounds heal quickly, scar contractures are slight after healing, and the composition is safe, reliable and free of toxic and side effects.

CN 104984078 A relates to traditional Chinese medicine, and particularly relates to a traditional Chinese medicine composition for treating qi-blood deficiency bedsores and a preparing method of the traditional Chinese medicine composition. The traditional Chinese medicine composition is composed of oral administration medicine and external use medicine. The oral administration medicine comprises Astragalus membranaceus, angelica sinensis, lentinus lepideus fr, fructus psoraleae, prepared rehmannia roots, schisandra propinqua, pseudo-ginseng, geranium wilfordii, ligusticum wallichii, cassia twig, sweetgum fruit, codonopsis pilosula and rhizoma atractylodis macrocephalae. The external use medicine comprises coda di volpe, herb of denseleaf crazyweed, beewax, caragana jubata, leaves of Chinese ilex, dwarf mallow and bletilla striata. The external use herb is absorbed by the skin and the ulcer surface, the disease resisting and defending capability is enhanced, the seepage of fester of the open sore is increased, the ulcer surface is kept wet, and therefore the functions of promoting the growth union of the ulcer surface and reducing scars are achieved; the traditional Chinese medicine composition is matched with the traditional Chinese medicine warming qi and blood, strengthening body resistance, dispersing blood stasis, activating blood and promoting granulation, inner and outer application is achieved, deficiency is supplemented, cold is scattered, stasis is released, and the effects of treating both symptoms and root causes are achieved for the treatment of the bedsores.

CN 104984073 A relates to traditional Chinese medicine, and particularly relates to a traditional Chinese medicine composition for treating second-stage bedsore in clinical nursing. The traditional Chinese medicine composition is prepared with asarum herb, bletilla striata, catechu, herba pteridis multifidae, typhonium divaricatum, mallotus repandus and other traditional Chinese herbal medicines as raw materials and is repeatedly verified by clinical tests; the traditional Chinese medicine composition directly acts on wound surface lesions, pus toxicity accumulated in sores can be early expelled out, slough can quickly fall off, and newborn granulation tissues are promoted to grow, wound healing is accelerated, the theory of traditional Chinese medicine is met, and the characteristics of traditional Chinese medicine external treatment is fully reflected. The formula has the advantages of good curative effect, convenient use, no pain, and low cost, and can be easily accepted by patients, and easily cures bedsore, especially early second-stage bedsore.

KR 101530574 B1 relates to a composition for a pressure ulcer and burn treatment, and more particularly, to silicon dioxide, aluminum oxide, iron oxide, titanium dioxide, calcium oxide, magnesium oxide, sodium oxide, and potassium oxide.

U.S. Pat. No. 8,591,960 B2 relates to a composition and method to prevent and treat bedsores rapidly, efficaciously and economically. The composition comprises a novel combination of herbal extracts that has surprisingly synergistic and highly efficacious effects for the acceleration of wound healing, and which also disinfects and relieves pain in dermal wounds. The composition can also be used for the rapid healing of dermal bums and dermal cuts. The composition is comprised of extracts of aloe vera, passion flower, goldenseal, and American ginseng, and optionally includes ionic silver and mimosa extract. The treatment method comprises administering a fluid and or gel form of the composition to the wounded skin and or wound cavity therein as a prophylactic or therapeutic dose, and may be delivered by means of a wound dressing.

Various compositions exist for preventing bed sores. However, their composition is substantially different from the present disclosure, as the other inventions fail to solve all the problems taught by the present disclosure.

### SUMMARY OF THE EMBODIMENTS

The present invention and its embodiments relate to a composition to prevent bed sores and a method to generate the composition.

A first embodiment of the instant invention provides a composition for external use. The composition is used to heal wounds, such as bedsores, pressure ulcers, lesions, cuts, and bums. In examples, the composition is used to prevent wounds, such as bedsores.

The composition includes: a thickening agent, an anti-fungal agent, an emulsifier, a glycerol, a skin protectant, a petroleum jelly, an oil, and at least one vitamin. The thickening agent is carbomer powder and is present in an amount of approximately 470 mL. The anti-fungal agent is a methylparben power and is present in an amount of approximately 240 mL. The emulsifier is cetearyl glucoside and is present in an amount of approximately 240 mL. The skin protectant is zinc oxide and is present in an amount of approximately 1420 mL. The amount of the glycerol is approximately 350 mL. The oil is a soybean oil and is present in an amount of approximately 240 mL. The at least one vitamin includes vitamin E and vitamin B. An amount of the at least one vitamin is approximately 590 mL. Further, the amount of the petroleum jelly is approximately 240 mL.

A ratio of the anti-fungal agent to the emulsifier to the oil and to the petroleum jelly is 1:1:1:1. Moreover, a ratio of the anti-fungal agent, the emulsifier, the oil, or the petroleum jelly to the skin protectant is 1:6. A ratio of the anti-fungal agent, the emulsifier, the oil, or the petroleum jelly to the thickening agent is 1:2. Additionally, a ratio of the anti-fungal agent, the emulsifier, the oil, or the petroleum jelly to the at least one vitamin is 1:2.5. Further, a ratio of the anti-fungal agent, the emulsifier, the oil, or the petroleum jelly to the glycerol is 1:1.5.

A second embodiment of the instant invention describes a method to create a composition for external use to prevent bedsores. The method includes: mixing a thickening agent, an anti-fungal agent, an emulsifier, a glycerol, a skin protectant, a petroleum jelly, an oil, and at least one vitamin to create the composition; and applying the composition to skin of a user. The user is a human.

In general, the present invention succeeds in conferring the following benefits and objectives.

It is an object of the present invention to provide a composition for human external use that prevents wounds.

It is an object of the present invention to provide a composition for human external use that heals wounds.

It is an object of the present invention to provide a composition for human external use that heals bedsores.

It is an object of the present invention to provide a composition for human external use that heals pressure ulcers.

It is an object of the present invention to provide a composition for human external use that heals lesions.

It is an object of the present invention to provide a composition for human external use that heals cuts.

It is an object of the present invention to provide a composition for human external use that heals burns.

### BRIEF DESCRIPTION OF THE DRAWINGS

FIG. 1 depicts a block diagram of a method to create a composition for external use to prevent or heal wounds, in accordance with embodiments of the present invention.

FIG. 2 depicts another block diagram of a method to create a composition for external use to prevent or heal wounds, in accordance with embodiments of the present invention.

### DESCRIPTION OF THE PREFERRED EMBODIMENTS

The preferred embodiments of the present invention will now be described with reference to the drawings. Identical elements in the various figures are identified with the same reference numerals.

Reference will now be made in detail to each embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

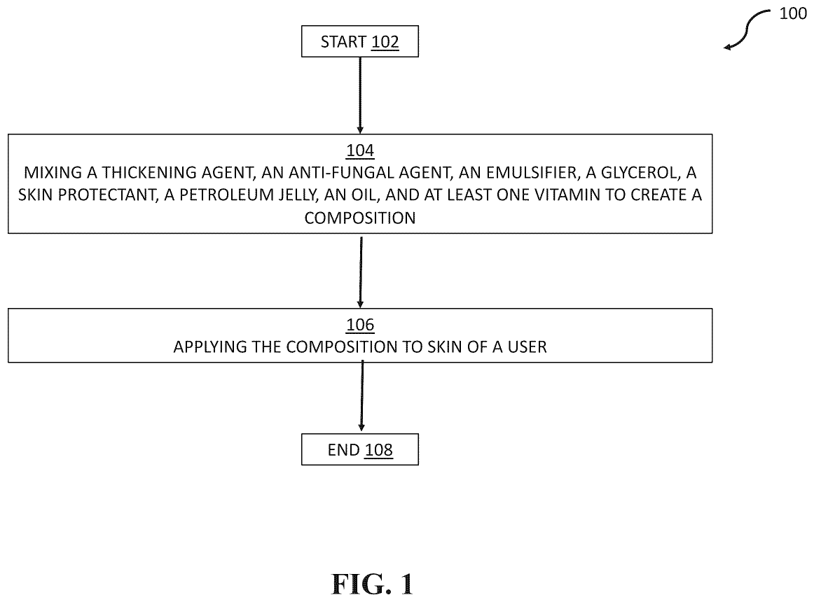

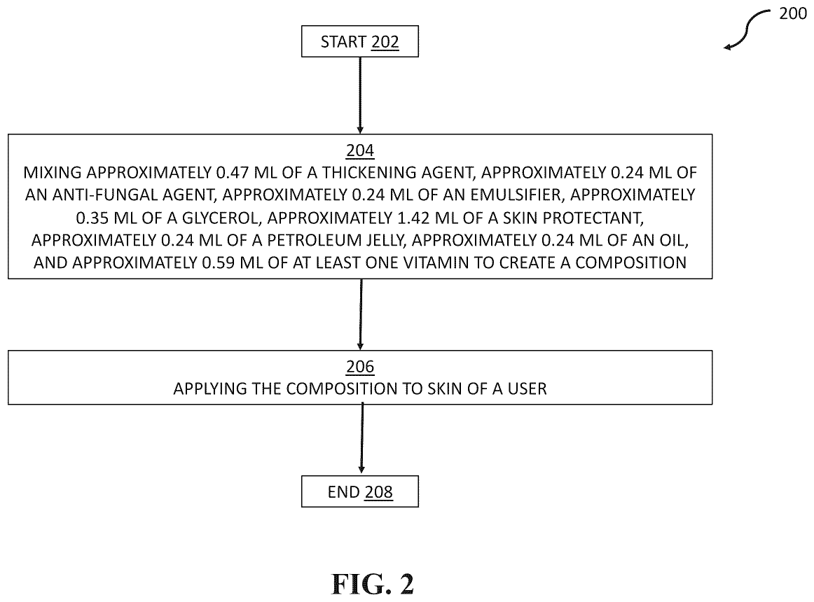

FIG. 1 and FIG. 2 depict block diagrams of methods to create a composition for external use to prevent or heal wounds, in accordance with embodiments of the present invention.

The process steps of a method 100 (of FIG. 1) to create a composition for external use to prevent or heal wounds begins at a process step 102. The process step 102 is followed by a process step 104 that involves mixing a thickening agent, an anti-fungal agent, an emulsifier, a glycerol, a skin protectant, a petroleum jelly, an oil, and at least one vitamin to create a composition.

In examples, the thickening agent is a carbomer powder, the anti-fungal agent is a methylparben power, the emulsifier is a cetearyl glucoside, the skin protectant is a zinc oxide, the oil is a soybean oil, and the at least one vitamin includes vitamin E and/or vitamin B.

The process step 104 is followed by a process step 106 that involves applying the composition to skin of a user. The user is a human user. The process step 106 is followed by a process step 108 that ends or concludes the method 100 to create the composition for external use to prevent or heal wounds. The wounds may include bedsores, pressure ulcers, lesions, cuts, and/or burns, among other wounds not explicitly listed herein.

The process steps of a method 200 (of FIG. 2) to create the composition for external use to prevent or heal wounds begins at a process step 202. The process step 202 is followed by a process step 204 that involves mixing approximately 470 mL (or 2 cups) of a thickening agent, approximately 240 mL (or 1 cup) of an anti-fungal agent, approximately 240 mL (or 1 cup) of an emulsifier, approximately 350 mL (or 1½ cups) of a glycerol, approximately 1420 mL (or 6 cups) of a skin protectant, approximately 240 mL (or 1 cup) of a petroleum jelly, approximately 240 mL (or 1 cup) of an oil, and approximately 590 mL (or 2½ cups) of at least one vitamin to create a composition. The process step 204 is followed by a process step 206 that involves applying the composition to skin of a user. The user is a human user. The process step 206 is followed by a process step 208 that ends or concludes the method 200 to create the composition for external use to prevent or heal wounds. The wounds may include bedsores, pressure ulcers, lesions, cuts, and/or burns, among other wounds not explicitly listed herein.

When introducing elements of the present disclosure or the embodiments thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements.

Although this invention has been described with a certain degree of particularity, it is to be understood that the present disclosure has been made only by way of illustration and that numerous changes in the details of construction and arrangement of parts may be resorted to without departing from the spirit and the scope of the invention.

What is claimed is:

1. A composition for external use, the composition comprising:
a thickening agent;
an anti-fungal agent;
an emulsifier;
a glycerol;
a skin protectant;
a petroleum jelly;
an oil; and
at least one vitamin.

2. The composition of claim 1, wherein the thickening agent is carbomer powder, and wherein an amount of the thickening agent is approximately 470 mL.

3. The composition of claim 1, wherein the anti-fungal agent is a methylparben power, and wherein an amount of the anti-fungal agent is approximately 240 mL.

4. The composition of claim 1, wherein the emulsifier is cetearyl glucoside, and wherein an amount of the emulsifier is approximately 240 mL.

5. The composition of claim 1, wherein the skin protectant is zinc oxide, and wherein an amount of the skin protectant is approximately 1420 mL.

6. The composition of claim 1, wherein an amount of the glycerol is approximately 350 mL.

7. The composition of claim 1, wherein the oil is a soybean oil, and wherein an amount of the oil is approximately 240 mL.

8. The composition of claim 1, wherein the at least one vitamin is selected from the group consisting of vitamin E and vitamin B, and wherein an amount of the at least one vitamin is approximately 590 mL.

9. The composition of claim 1, wherein an amount of the petroleum jelly is approximately 240 mL.

10. The composition of claim 1, wherein a ratio of the anti-fungal agent to the emulsifier to the oil and to the petroleum jelly is 1:1:1:1.

11. The composition of claim 1, wherein a ratio of the anti-fungal agent, the emulsifier, the oil, or the petroleum jelly to the skin protectant is 1:6.

12. The composition of claim 1, wherein a ratio of the anti-fungal agent, the emulsifier, the oil, or the petroleum jelly to the thickening agent is 1:2.

13. The composition of claim 1, wherein a ratio of the anti-fungal agent, the emulsifier, the oil, or the petroleum jelly to the at least one vitamin is 1:2.5.

14. The composition of claim 1, wherein a ratio of the anti-fungal agent, the emulsifier, the oil, or the petroleum jelly to the glycerol is 1:1.5.

15. The composition of claim 1, wherein the composition is used to heal wounds.

16. The composition of claim 15, wherein the wounds are selected from the group consisting of: bedsores, pressure ulcers, lesions, cuts, and bums.

17. The composition of claim 1, wherein the composition is used to prevent wounds.

18. The composition of claim 17, wherein the wounds include bedsores.

19. A method to create a composition for external use to prevent bedsores, the method comprising:
mixing a thickening agent, an anti-fungal agent, an emulsifier, a glycerol, a skin protectant, a petroleum jelly, an oil, and at least one vitamin to create the composition; and
applying the composition to skin of a user.

20. The method of claim 19, wherein the user is a human user.
